(19) United States
(12) Patent Application Publication
(10) Pub. No.: US 2009/0005815 A1
(43) Pub. Date: Jan. 1, 2009

(54) DYNAMIC STABILIZATION SYSTEM

(76) Inventor: SCOTT ELY, Cedar Park, TX (US)

Correspondence Address: PAUL D. YASGER;ABBOTT LABORATORIES, 100 ABBOTT PARK ROAD, DEPT. 377/AP6A, ABBOTT PARK, IL, 60064-6008, US

(21) Appl. No.: 11/770,542

(22) Filed: Jun. 28, 2007

(51) Int. Cl. A61B 17/70; A61B 17/58; A61B 17/04

(52) U.S. Cl. 606/246; 606/250; 606/264; 606/301; 606/305

(57) ABSTRACT

Embodiments of the disclosure provide a device and system for dynamic spinal stabilization. A collar has an opening for connection to a bone fastener implanted in bony tissue and a slot for receiving a portion of an elongated member. When the elongated member is seated in the slot and a closure member is securely connected to the collar, the collar is capable of selected rotation about the bone fastener to provide dampened motion of the stabilization system.

### FIELD OF THE DISCLOSURE

This disclosure relates generally to stabilizing movement between bony tissues within a body, and in particular to systems and methods for stabilizing movement between vertebral bodies. Even more particularly, embodiments of the present disclosure relate to systems and methods for dynamic stabilization of vertebral bodies.

### BACKGROUND OF THE DISCLOSURE

The human spine is a column of vertebrae separated by spinal discs that protects the spinal cord and various nerves and blood vessels and supports the torso. Under normal circumstances, the spine is capable of movement such as flexion, extension and torsion. However, in medical situations such as injury, trauma, stress, or disease (both acute and chronic) stabilization of at least a portion of the spine may be necessary for healing or therapeutic benefits. In particular, it is sometimes necessary to limit or control movement between two or more vertebrae, either temporarily or permanently.

Modern spine surgery often involves spinal fixation through the use of spinal implants or fixation systems to correct or treat various spine disorders or to support the spine. Spinal implants may help, for example, to stabilize the spine, correct deformities of the spine, facilitate fusion, or treat spinal fractures. A spinal fixation system typically includes corrective spinal instrumentation that is attached to selected vertebra of the spine by screws, hooks, and clamps. The corrective spinal instrumentation includes spinal rods or plates that are generally parallel to the patient's back. The corrective spinal instrumentation may also include transverse connecting elongated members that extend between neighboring spinal rods. Spinal fixation systems are used to correct problems in the cervical, thoracic, and lumbar portions of the spine, and are often installed posterior to the spine on opposite sides of the spinous process and adjacent to the transverse process.

Various types of screws, hooks, and clamps have been used for attaching corrective spinal instrumentation to selected portions of a patient's spine.

Often, spinal fixation may include rigid (i.e., in a fusion procedure) support for the affected regions of the spine. Such systems limit movement in the affected regions in virtually all directions (for example, in a fused region). More recently, so called “dynamic” systems have been introduced wherein the implants allow at least some movement of the affected regions in at least some directions, i.e. flexion, extension, lateral, or torsional.

Prior art spinal stabilization systems run the risk that a rod too rigid to support the spine across the injured or degenerative disk will prevent desired degree of flexion, and may overcompensate by overextending, overflexing, or overtorquing the spine at adjacent vertebrae, which can result in discomfort, pain, injury, or degenerative effects. Prior art spinal stabilization systems also run the risk that an elongated member is too flexible to support the spine across the injured or degenerative disk and will prevent the disk from healing properly, resulting in discomfort, pain, or decreased functionality. In other words, if the stabilization system is too rigid, the stresses may be transferred to the adjacent vertebrae, resulting in additional injured or damaged disks, but if the rod is too flexible, the system may provide insufficient support, resulting in poor alignment, pain, longer healing times, or other undesirable effects.

### SUMMARY OF THE DISCLOSURE

Embodiments of the present disclosure provide dynamic stabilization to facilitate recovery from spinal injuries and degenerative conditions. To achieve this and other goals, embodiments of the present disclosure have anchors implanted in vertebral bodies and an elongated member attached to the bone fasteners such that the elongated member can rotate relative to the bone fasteners. The resulting system allows for more natural movement of the spine.

Unlike prior art bone fastener assemblies that fixed the vertebrae, embodiments of the present disclosure may be used to preserve movement between vertebrae in desired planes. Bone fasteners may be implanted in the pedicle of vertebral bodies. Friction reducing members may be positioned about the head of the bone fastener. Collars may be attached to the bone fasteners with the friction reducing member interposed, and an elongated member may be attached to connect two or more collars. Collars, friction reducing members, and/or bone fasteners may be configured to have a low friction coefficient. Vertebral bodies are able to move through some range because the elongated member can rotate relative to the collar, thus improving the range of motion of the spine without damaging adjacent vertebrae.

Although this embodiment realizes advantages in attaching anchors to pedicles, the present disclosure is not limited. The present disclosure enables the implantation of a dynamic stabilization system to lateral or other desired sites for use in the correction of spinal conditions, with each having various advantages, such as degrees of motion in a desired plane. Furthermore, embodiments of the present disclosure may be used in cooperation with flexible or dynamic elongated members to construct a dynamic stabilization system with a generic component (i.e. an elongated member having homogeneous properties such as torsional stiffness) and a particular component (i.e. a collar in combination with a selected friction reducing member such as a swivel bearing, stationary bearing, compression bearing, or a bone fastener having a layer of UHMWPE) individually selected based on the patient's needs.

In some embodiments, the present disclosure is generally directed to dynamic stabilization collar comprising an opening for receiving a portion of a bone fastener and a slot at least partially open to the opening and configured to receive at least a portion of an elongated member. A closure member may be configured for selected contact with the elongated member and further configured for secure connection to the collar to retain the elongated member in the slot. A friction reducing member disposed between at least a portion of the opening and at least a portion of the bone fastener may be configured to provide selected contact, such that dampened polyaxial motion of the elongated member relative to the bone fastener is preserved when the closure member is securely connected to the collar. The friction reducing member may be a swivel bearing having an inner surface configured for rotatable contact with the bone fastener and an outer surface configured for polyaxial contact between the collar and the swivel bearing. The friction reducing member may be a stationary bearing having an outer surface configured for polyaxial motion relative to the inner surface of the collar and an inner surface configured for polyaxial motion relative to the bone fastener. The friction reducing member may be a compression bearing comprising an upper portion and a lower portion, and the inner surface of the upper and lower portions may be configured for polyaxial motion between the bone fastener and the compression bearing, and an outer surface configured for rotatable contact between the collar and the compression bearing. The collar may also have a channel having a cylindrical inner surface with modified thread portion to engage a closure member configured with a helically wound thread. The closure member may have a layer having a low friction coefficient for selected contact such as reduced friction with the elongated member. A portion of the collar, swivel bearing, stationary bearing, compression bearing, or bone fastener may include a layer of UHMWPE, PEEK, or other friction reducing materials.

Another embodiment is directed to a dynamic stabilization system, having two or more bone fasteners implantable in bony tissue, an elongated member of selected length to span between the two or more bone fasteners implanted in bony tissue, and two or more collars for connecting the elongated member to the two or more bone fasteners. Each collar may include an opening for receiving a portion of a bone fastener. Each collar may include a slot at least partially open to the opening and configured to receive at least a portion of an elongated member. A closure member configured for selected contact with the elongated member and further configured for secure connection to the collar to retain the elongated member in the slot may be connected to the collar. A friction reducing member disposed between at least a portion of the opening and at least a portion of the bone fastener may be configured to provide selected friction resistance, such that dampened polyaxial motion of the elongated member relative to the bone fastener is preserved when the closure member is securely connected to the collar. The friction reducing member may be a swivel bearing having an inner surface configured for rotatable contact with the bone fastener and an outer surface configured for polyaxial contact between the collar and the swivel bearing. The friction reducing member may be a stationary bearing having an outer surface configured for polyaxial contact relative to the inner surface of the collar and an inner surface configured for polyaxial contact relative to the bone fastener. The friction reducing member may be a compression bearing comprising an upper portion and a lower portion, wherein the inner surface of the upper and lower portions is configured for polyaxial motion between the bone fastener and the compression bearing, and further comprising an outer surface configured for rigid contact between the collar and the compression bearing. The collar may also have a channel having a cylindrical inner surface with modified thread portion to engage a closure member configured with a helically wound thread. The closure member may have a layer having a low friction coefficient for selected contact such as reduced friction with the elongated member. A portion of the collar, swivel bearing, stationary bearing, compression bearing, or bone fastener may include a layer of UHMWPE.

Yet another embodiment is directed to a method for dynamically stabilizing a spine by coupling a collar to an bone fastener in a bony tissue, positioning a portion of a elongated member in the collar, and connecting the closure member to the collar to maintain the collar in movable contact with the bone fastener. The collar may be configured such that dampened polyaxial motion of the elongated member relative to the bone fastener is preserved when the closure member is securely connected to the collar. The step of connecting the closure member to the collar to maintain the collar in selected contact with the bone fastener may be achieved by rotatably engaging the closure member with a modified thread portion, wherein the closure member is configured with a helically wound thread. The method may further include the step of positioning a friction reducing member inside the collar for low friction coefficient.

These, and other, aspects of the disclosure will be better appreciated and understood when considered in conjunction with the following description and the accompanying drawings. The following description, while indicating various embodiments of the disclosure and numerous specific details thereof, is given by way of illustration and not of limitation. Many substitutions, modifications, additions or rearrangements may be made within the scope of the disclosure, and the disclosure includes all such substitutions, modifications, additions or rearrangements.

### BRIEF DESCRIPTION OF THE DRAWINGS

A more complete understanding of the present disclosure and the advantages thereof may be acquired by referring to the following description, taken in conjunction with the accompanying drawings in which like reference numbers indicate like features and wherein:

FIG. 1 depicts a perspective view of an embodiment of a spinal stabilization system.

FIG. 2 depicts a perspective view of an embodiment of a bone fastener assembly.

FIG. 3 depicts a perspective view of an embodiment of a bone fastener.

FIG. 4A depicts a swivel bearing embodiment of a friction reducing member useful for preserving polyaxial motion of an elongated member relative to a bone fastener.

FIG. 4B depicts a stationary bearing embodiment of a friction reducing member useful for preserving polyaxial motion of an elongated member relative to a bone fastener.

FIG. 4C depicts a compression bearing embodiment of a friction reducing member useful for preserving polyaxial motion of an elongated member relative to a bone fastener.

FIG. 5 depicts a perspective view of an embodiment of a bone fastener assembly collar.

FIG. 6 depicts a cross-sectional view of an embodiment of a bone fastener assembly.

FIGS. 7A-7C depict schematic views of a method of positioning a swivel bearing in a collar of a bone fastener assembly.

FIGS. 8A-8C depict schematic views of a method of positioning a swivel bearing in a collar of a bone fastener assembly.

FIG. 9 depicts a front view of an embodiment of a bone fastener assembly with a collar that allows for angulation of a bone fastener relative to the collar in a conical range of motion that is symmetrical relative to an axis that passes through a central axis of the collar and a central axis of a bone fastener.

FIG. 10A depicts a front view of an embodiment of a bone fastener assembly with a collar that allows for angulation of a bone fastener relative to the collar in a conical range of motion that is not symmetrical relative to an axis that passes through a central axis of the collar and a central axis of a bone fastener. The collar allows additional lateral bias relative to a non-biased collar.

FIG. 10B depicts a side view of an embodiment of a bone fastener assembly with a collar that allows for angulation of a bone fastener relative to the collar in a conical range of motion that is not symmetrical relative to an axis that passes through a central axis of the collar and a central axis of a bone fastener. The collar allows additional caudal or cephalid bias relative to a non-biased collar.

FIG. 11A depicts a schematic side view representation of embodiments of bone fastener assemblies positioned in vertebrae.

FIG. 11B depicts a schematic top view representation of an embodiment of a single-level spinal stabilization system.

FIG. 12 depicts a perspective view of an embodiment of a closure member.

FIG. 13 depicts a cross-sectional representation of the closure member taken substantially along plane 15-15 indicated in FIG. 12.

FIG. 14 depicts a perspective view of an embodiment of a portion of a spinal stabilization system.

FIG. 15A depicts a cross-sectional representation of an embodiment of a spinal stabilization system.

FIG. 15B depicts a detailed view of a portion of FIG. 15A.

FIG. 16 depicts a perspective view of a bone fastener used in an invasive procedure.

### DETAILED DESCRIPTION

The disclosure and the various features and advantageous details thereof are explained more fully with reference to the non-limiting embodiments that are illustrated in the accompanying drawings and detailed in the following description. Descriptions of well known starting materials, processing techniques, components and equipment are omitted so as not to unnecessarily obscure the disclosure in detail. Skilled artisans should understand, however, that the detailed description and the specific examples, while disclosing preferred embodiments of the disclosure, are given by way of illustration only and not by way of limitation. Various substitutions, modifications, additions or rearrangements within the scope of the underlying inventive concept(s) will become apparent to those skilled in the art after reading this disclosure.

Reference is now made in detail to the exemplary embodiments of the disclosure, examples of which are illustrated in the accompanying drawings. Wherever possible, the same reference numbers will be used throughout the drawings to refer to the same or like parts (elements.)

The systems and methods of the disclosure may be particularly useful for stabilizing movement in the spine and thus it is in this context that embodiments of the disclosure may be described. It will be appreciated, however, that embodiments of the devices and systems of the present disclosure may be applicable for stabilizing movement in other areas of the spine or body.

One of the reasons that embodiments of the present disclosure may be usefully applied to stabilize movement in the spine is the ability to control rotation of the collar about the bone fastener to allow movement of vertebrae, thereby improving the stabilization system.

For purposes of this document, the terms stabilize and stabilization generally refer to the control of one or more degrees of freedom for movement. Rotation, flexion and extension are examples of movement that may be controlled using a spinal stabilization system. Stabilization may result in the complete restriction of movement about a particular axis or plane, or it may limit the movement over a selected range of motion.

A spinal stabilization system may be installed in a patient to stabilize a portion of a spine. Spinal stabilization may be used, but is not limited to use, in patients having degenerative disc disease, spinal stenosis, spondylolisthesis, pseudoarthrosis, and/or spinal deformities; in patients having fracture or other vertebral trauma; and in patients after tumor resection. A spinal stabilization system may be installed using a minimally invasive procedure. An instrumentation set may include instruments and spinal stabilization system components for forming a spinal stabilization system in a patient.

A minimally invasive procedure may be used to limit an amount of trauma to soft tissue surrounding vertebrae that are to be stabilized. In some embodiments, the natural flexibility of skin and soft tissue may be used to limit the length and/or depth of an incision or incisions needed during the stabilization procedure. Minimally invasive procedures may provide limited direct visibility in vivo. Forming a spinal stabilization system using a minimally invasive procedure may include using tools to position system components in the body.

A minimally invasive procedure may be performed after installation of one or more spinal implants in a patient. The spinal implant or spinal implants may be inserted using an anterior procedure and/or a lateral procedure. The patient may be turned and a minimally invasive procedure may be used to install a posterior spinal stabilization system. A minimally invasive procedure for stabilizing the spine may be performed without prior insertion of one or more spinal implants in some patients. In some patients, a minimally invasive procedure may be used to install a spinal stabilization system after one or more spinal implants are inserted using a posterior spinal approach.

A spinal stabilization system may be used to achieve rigid pedicle fixation while minimizing the amount of damage to surrounding tissue. In some embodiments, a spinal stabilization system may be used to provide stability to two adjacent vertebrae (i.e., one vertebral level). A spinal stabilization system may include two bone fastener assemblies. One bone fastener assembly may be positioned in each of the vertebrae to be stabilized. An elongated member may be coupled and secured to the bone fastener assemblies. As used herein, “coupled” components may directly contact each other or may be separated by one or more intervening members. In some embodiments, a single spinal stabilization system may be installed in a patient. Such a system may be referred to as a unilateral, single-level stabilization system or a single-level, two-point stabilization system. In some embodiments, two spinal stabilization systems may be installed in a patient on opposite sides of a spine. Such a system may be referred to as a bilateral, single-level stabilization system or a single-level, four-point stabilization system.

In some embodiments, a spinal stabilization system may provide stability to three or more vertebrae (i.e., two or more vertebral levels). In a two vertebral level spinal stabilization system, the spinal stabilization system may include three bone fastener assemblies. One bone fastener assembly may be positioned in each of the vertebrae to be stabilized. An elongated member may be coupled and secured to the three bone fastener assemblies. In some embodiments, a single two-level spinal stabilization system may be installed in a patient. Such a system may be referred to as a unilateral, two-level stabilization system or a two-level, three-point stabilization system. In some embodiments, two three-point spinal stabilization systems may be installed in a patient on opposite sides of a spine. Such a system may be referred to as a bilateral, two-level stabilization system or a two-level, six-point stabilization system.

In some embodiments, combination systems may be installed. For example, a two-point stabilization system may be installed on one side of a spine, and a three-point stabilization system may be installed on the opposite side of the spine. The composite system may be referred to a five-point stabilization system.

Minimally invasive procedures may reduce trauma to soft tissue surrounding vertebrae that are to be stabilized. Only a small opening may need to be made in a patient. For example, for a single-level stabilization procedure on one side of the spine, the surgical procedure may be performed through a 2 cm to 4 cm incision formed in the skin of the patient. In some embodiments, the incision may be above and substantially between the vertebrae to be stabilized. In some embodiments, the incision may be above and between the vertebrae to be stabilized. In some embodiments, the incision may be above and substantially halfway between the vertebrae to be stabilized. Dilators, a targeting needle, and/or a tissue wedge may be used to provide access to the vertebrae to be stabilized without the need to form an incision with a scalpel through muscle and other tissue between the vertebrae to be stabilized. A minimally invasive procedure may reduce an amount of post-operative pain felt by a patient as compared to invasive spinal stabilization procedures. A minimally invasive procedure may reduce recovery time for the patient as compared to invasive spinal procedures.

Components of spinal stabilization systems may be made of materials including, but not limited to, titanium, titanium alloys, stainless steel, ceramics, and/or polymers. Some components of a spinal stabilization system may be autoclaved and/or chemically sterilized. Components that may not be autoclaved and/or chemically sterilized may be made of sterile materials. Components made of sterile materials may be placed in working relation to other sterile components during assembly of a spinal stabilization system.

Spinal stabilization systems may be used to correct problems in lumbar, thoracic, and/or cervical portions of a spine. Various embodiments of a spinal stabilization system may be used from the C1 vertebra to the sacrum. For example, a spinal stabilization system may be implanted posterior to the spine to maintain distraction between adjacent vertebral bodies in a lumbar portion of the spine.

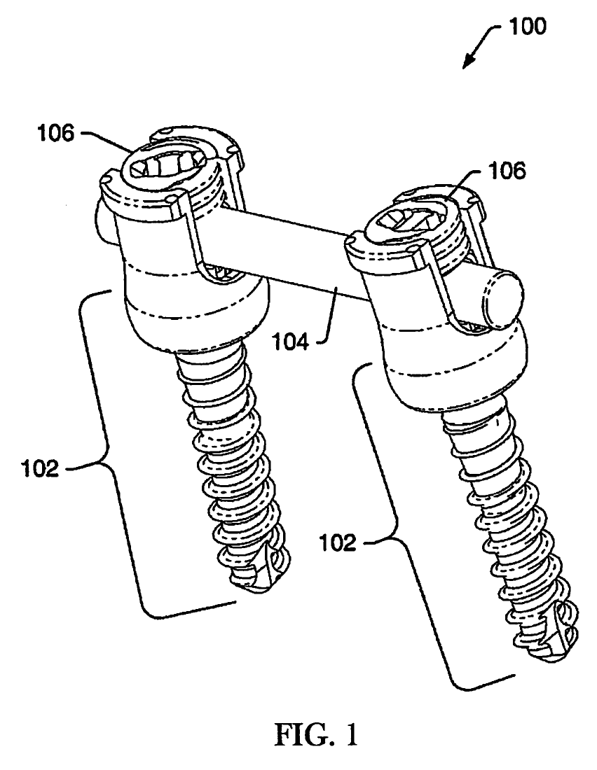

FIG. 1 depicts an embodiment of spinal stabilization system 100 that may be implanted using a minimally invasive surgical procedure. Spinal stabilization system 100 may include bone fastener assemblies 102, elongated member 104, and/or closure members 106. Other spinal stabilization system embodiments may include, but are not limited to, plates, dumbbell-shaped members, and/or transverse connectors. FIG. 1 depicts a spinal stabilization system for one vertebral level. In some embodiments, the spinal stabilization system of FIG. 1 may be used as a multi-level spinal stabilization system if one or more vertebrae are located between the vertebrae in which bone fastener assemblies 102 are placed. In other embodiments, multi-level spinal stabilization systems may include additional bone fastener assemblies to couple to one or more other vertebrae.

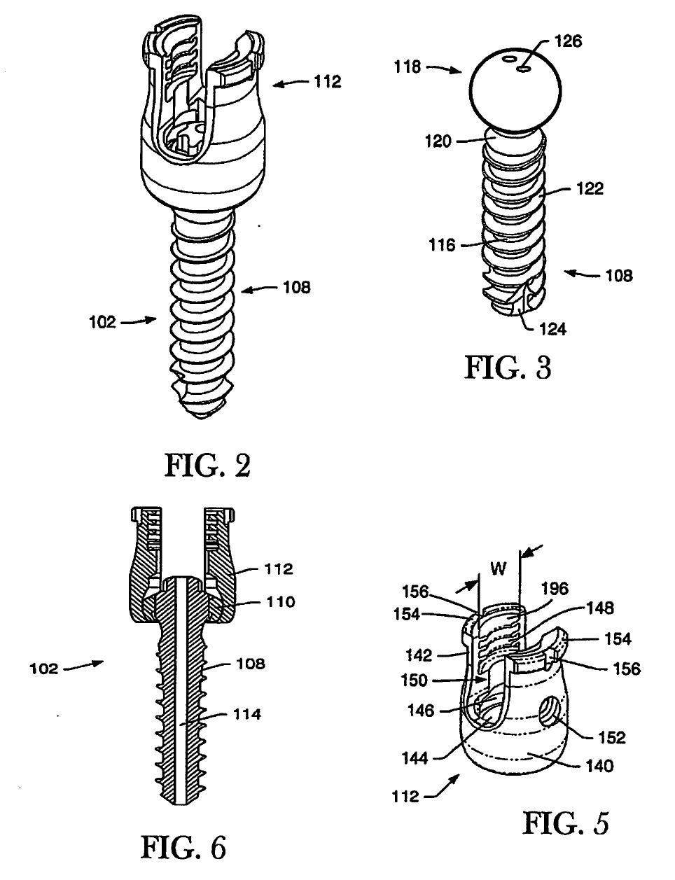

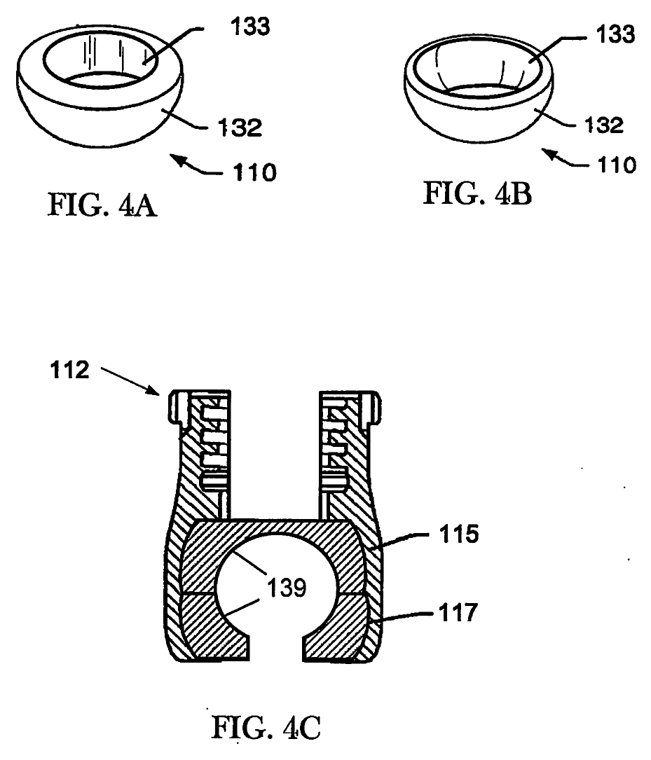

FIG. 2 depicts a perspective view of bone fastener assembly 102. FIGS. 3, 4A, 4B, 4C and 5 depict embodiments of bone fastener assembly components. Components of bone fastener assembly 102 may include bone fastener 108 (shown in FIG. 3). Bone fastener 108 may couple bone fastener assembly 102 to a vertebra. Bone fastener assembly 102 may include a friction reducing member such as swivel bearing 110 (shown in FIG. 4A), stationary bearing 123 (shown in FIG. 4B, and compression bearing 113 (shown in FIG. 4C). A friction reducing member may reduce the friction force in a plane, about an axis, or about multiple planes or axes to enable polyaxial motion. Bone fastener assembly 102 may include a collar 112 (shown in FIG. 5) for coupling an elongated rod to bone fastener assembly 102.

A bone fastener may be, but is not limited to, a bone screw, a ring shank fastener, a barb, a nail, a brad, or a trocar. Bone fasteners and/or bone fastener assemblies may be provided in various lengths in an instrumentation set to accommodate variability in vertebral bodies. For example, an instrumentation set for stabilizing vertebrae in a lumbar region of the spine may include bone fastener assemblies with lengths ranging from about 30 mm to about 75 cm in 5 mm increments. A bone fastener assembly may be stamped with indicia (i.e., printing on a side of the collar). In some embodiments, a bone fastener assembly or a bone fastener may be color-coded to indicate a length of the bone fastener. In certain embodiments, a bone fastener with a 30 cm thread length may have a magenta color, a bone fastener with a 35 cm thread length may have an orange color, and a bone fastener with a 55 mm thread length may have a blue color. Other colors may be used as desired.

Each bone fastener provided in an instrumentation set may have substantially the same thread profile and thread pitch. In an embodiment, the thread may have about a 4 cm major diameter and about a 2.5 mm minor diameter with a cancellous thread profile. In certain embodiments, the minor diameter of the thread may be in a range from about 1.5 mm to about 4 cm or larger. In certain embodiments, the major diameter of the thread may be in a range from about 3.5 mm to about 6.5 mm or larger. Bone fasteners with other thread dimensions and/or thread profiles may also be used. A thread profile of the bone fasteners may allow bone purchase to be maximized when the bone fastener is positioned in vertebral bone.

FIG. 3 depicts an embodiment of bone fastener 108. Bone fastener 108 may include shank 116, head 118, and neck 120. Shank 116 may include threading 122. In some embodiments, threading 122 may include self-tapping start 124. Self-tapping start 124 may facilitate insertion of bone fastener 108 into vertebral bone. In some embodiments, bone fastener 108 may be cannulated for use in a minimally invasive procedure.

Head 118 of bone fastener 108 may have a spherical surface, as depicted in FIG. 3. In some head embodiments, head 118 may include a layer configured for selected contact (such as reduced friction) with a collar, or for selected contact with a friction reducing member to reduce friction. Head 118 of bone fastener 108 may include various configurations to engage a driver that inserts the bone fastener into a vertebra. In some embodiments, the driver may also be used to remove an installed bone fastener from a vertebra. In some embodiments, head 118 may include one or more tool portions 126. Tool portions 126 may be recesses and/or protrusions designed to engage a portion of the driver.

Neck 120 of bone fastener 108 may have a smaller diameter than adjacent portions of head 118 and shank 116. The diameter of neck 120 may fix the maximum angle that the collar of the bone fastener assembly can be rotated relative to bone fastener 108. In some embodiments, neck 120 may be sized to allow up to about 40 degree or more of angulation of the collar relative to the bone fastener. In some embodiments, the neck may be sized to allow up to about 30 degree of angulation of the collar relative to the bone fastener. In some embodiments, the neck may be sized to allow up to about 20 degrees of angulation of the collar relative to the bone fastener.

FIG. 4A depicts a perspective view of an embodiment of a swivel bearing 110 that may function as a friction reducing member in a bone fastener assembly. Outer surface 132 of swivel bearing 110 may be configured with a polished or otherwise smooth finish such that contact with a collar has a low friction coefficient. In some embodiments, outer surface 132 may be surface treated or include coatings and/or coverings. Surface treatments, coatings, and/or coverings may be used to adjust frictional and/or wear properties of the outer surface of the swivel bearing.

Outer surface 132 of swivel bearing 110 may be configured to contour an inner surface of a collar in which the bearing resides. A contour of the outer surface of swivel bearing 110 may be a spherical portion. The contour of the outer surface 132 of the swivel bearing 110 and the inner surface of the collar 112 may inhibit removal of the swivel bearing 110 from the collar 112 after insertion of the swivel bearing 110 into the collar 112. When swivel bearing 110 is positioned in the collar, the complementary shape of the swivel bearing 110 outer surface 132 and the inner surface of the collar 112 that contacts the swivel bearing 110 may allow angulation of a collar relative to a bone fastener 108 coupled to the swivel bearing 110. In some embodiments, a portion of the outer surface of the bearing may be shaped and/or textured to limit a range of motion of the collar relative to a bone fastener of a bone fastener assembly.

Outer surface 132 of swivel bearing 110 may further be configured for selected contact with collar 112. Outer surface 132 may be manufactured from UHMWPE, PEEK, or other material having low friction coefficient.

Inner surface 133 of swivel bearing 110 may be configured to contour to an outer surface of a bone fastener such as bone fastener 108 in FIG. 2. An inner surface 133 of swivel bearing 110 may include one or more surfaces for selected contact with one or more surfaces on the head of a bone fastener. A surface of the inner surface 133 of the swivel bearing 110 may be a cylindrical portion. When the swivel bearing 110 is positioned about a bone fastener, the contact between the swivel bearing inner surface 133 and the bone fastener outer surface may allow rotation of the collar relative to a bone fastener. In embodiments in which inner surface 133 of swivel bearing 110 has a cylindrical form corresponding with a cylindrical portion of a head of a bone fastener, rotatable contact about a selected plane or about a selected axis is possible. In embodiments in which inner surface 133 of swivel bearing 110 has a spherical form corresponding with a spherical portion of a bone fastener, polyaxial contact may allow rotation in any plane or about any axis.

Inner surface 133 of swivel bearing 110 may further be configured for selected contact with bone fastener based on materials. Inner surface 133 may be manufactured from UHMWPE, PEEK, or other material having low friction coefficient. Inner surface 133 of swivel bearing 110 may have a polished or otherwise smooth finish. In some embodiments, inner surface 133 may be surface treated or include coatings and/or coverings. Surface treatments, coatings, and/or coverings may be used to adjust frictional and/or wear properties of the inner surface 133 of the swivel bearing 110. In some embodiments inner surface 133 may be configured with a layer of UHMWPE or PEEK to provide a low friction coefficient for reduced friction. In some embodiments, a portion of the inner surface 133 of the swivel bearing 110 may be shaped and/or textured to limit a range of motion of the collar 112 relative to a bone fastener of a bone fastener assembly.

In some embodiments, not shown, a swivel bearing may be a complete bearing without a split or slots. In some embodiments, a swivel bearing 110 may include a split or slots to facilitate insertion of the swivel bearing into a collar. In some embodiments, a swivel bearing with a split and/or slots may be compressed to ease insertion into a collar. Once positioned in the collar, the swivel bearing may expand to its original uncompressed dimensions, thus inhibiting removal from the collar.

In some embodiments, head of bone fastener 108 may be polished or otherwise treated to have a low friction coefficient. In some embodiments, a layer may be applied to the head of a bone fastener to provide a low friction coefficient.

FIG. 4B depicts a perspective view of an embodiment of a friction reducing member which may be useful as a friction reducing member. Stationary bearing 111 may include an outer surface 135 and an inner surface 137.

Outer surface 135 of stationary bearing 111 may be configured to contour to a portion of an inner surface of a collar in which the stationary bearing 111 resides. A contour of the outer surface 135 of the stationary bearing 111 may be a spherical portion. When the stationary bearing 111 is positioned in the collar 112, the contact between the stationary bearing 111 outer surface 135 and the collar 112 inner surface (such as inner surface 146 in FIG. 5) may allow angulation of the collar 112 relative to the stationary bearing 111. The contours of the outer surface 135 of the stationary bearing 111 and the inner surface of the collar 112 may inhibit removal of the stationary bearing 111 from the collar 112 after insertion of the stationary bearing 111 into the collar 112. Outer surface 135 of stationary bearing 111 may further be configured for selected contact with a portion of a collar based on materials. Outer surface 135 may be manufactured from UHMWPE, PEEK, or other material having low friction coefficient.

Inner surface 137 of stationary bearing 111 may be configured to contour to a portion of a bone fastener 108 about which the stationary bearing 111 resides. A contour of the inner surface 137 of the stationary bearing 111 may be a spherical portion for polyaxial contact with a spherical head of a bone fastener as shown in FIG. 2.

When the stationary bearing 111 is positioned in the collar 112, the complementary shape and contact between the inner surface 137 of the stationary bearing 111 and the head of the bone fastener 108 may allow angulation of the stationary bearing 111 relative to a bone fastener 108. Alternatively, when the stationary bearing 111 is positioned in the collar 112, the complementary shape and contact between the outer surface 135 of the stationary bearing 111 and the inner surface 162 of collar 112 may allow angulation of the stationary bearing 111 relative to the collar. Alternatively, stationary bearing 111 may remain relatively fixed in position and collar 112 and bone fastener 108 may rotate relative each other due to the polyaxial contact between inner surface 135 and bone fastener 108 and/or outer surface 137 and inner surface 146 of collar 112. In other words, stationary bearing 185 may remain stationary and collar 112 may rotate about bone fastener 108 due to the polyaxial contact between inner surface 137 of stationary bearing 111 and a surface of the head of a bone fastener, or may rotate due to the polyaxial contact between outer surface 135 of stationary bearing 111 and an inner surface of a collar, or both.

In some embodiments, head of bone fastener 108 may be polished or otherwise treated to have a low friction coefficient. In some embodiments, a layer may be applied to the head of a bone fastener to provide a low friction coefficient. Inner surface 137 of stationary bearing 111 may have a polished or otherwise smooth finish. In some embodiments, inner surface 137 may be surface treated or include coatings and/or coverings. Surface treatments, coatings, and/or coverings may be used to adjust frictional and/or wear properties of the inner surface 137 of the stationary bearing 111. In some embodiments inner surface 137 may be configured with a layer of UHMWPE or PEEK to provide a low friction coefficient for reduced friction. In some embodiments, a portion of the inner surface 137 of the stationary bearing 111 may be shaped and/or textured to limit a range of motion of the collar 112 relative to a bone fastener of a bone fastener assembly.

In some embodiments, not shown, a stationary bearing may be a complete bearing without a split or slots. In some embodiments, a stationary bearing may include a split or slots to facilitate insertion of the stationary bearing into a collar. In some embodiments, a stationary bearing with a split and/or slots may be compressed to ease insertion into a collar. Once positioned in the collar, the stationary bearing may expand to its original uncompressed dimensions, thus inhibiting removal from the collar.

FIG. 4C depicts a perspective view of an embodiment of a compression bearing 113 having upper portion 115 and lower portion 117 that may be useful as a friction reducing member. In this configuration, upper portion 115 may be compressed onto lower portion 117 (such as by closure member 106 shown in FIG. 1). The compression may generate a desired friction force for dampened polyaxial motion such that compression bearing 113 remains stationary relative to collar 112 yet compression bearing 195 may exhibit polyaxial motion relative to a bone fastener (such as bone fastener 108 depicted in FIG. 2).

Upper portion 115 and lower portion 117 may be manufactured from materials such as UHMWPE, PEEK, or other polymers or ceramics selected for a low friction coefficient. In preferred embodiments, head of bone fastener 108 may be polished or otherwise treated to have a low friction coefficient.

Upper portion 115 and lower portion 117 of compression bearing 113 may have a contour that substantially complements a contour of an inner surface of a collar in which the compression bearing 113 resides. A contour of the upper portion 115 and lower portion 117 of the compression bearing 113 may have a spherical portion. When the compression bearing 113 is positioned in the collar 112, the complementary shape of the compression bearing 113 inhibits angulation of the collar 112 relative to the compression bearing 113. The contour of the upper portion 115 and lower portion 117 of the compression bearing 113 and the inner surface of the collar 112 may inhibit removal of the compression bearing 113 from the collar 112 after insertion of the compression bearing 113 into the collar 112. When the compression bearing 113 is positioned in the collar 112, the complementary shape of the compression bearing 113 inner surfaces 139 and the outer surface 141 of the bone fastener 108 that contacts the compression bearing 113 further allows angulation of the collar 112 relative to a bone fastener 108 coupled to the compression bearing 113.

Inner surfaces 139 of compression bearing 113 may have a smooth or polished finish. In some embodiments, inner surface 139 may be surface treated or include coatings and/or coverings, for example Ultra High Molecular Weight Polyethylene (UHMWPE). Surface treatments, coatings, and/or coverings may be used to adjust frictional and/or wear properties of the inner surfaces 139 of the compression bearing 113. In some embodiments, a portion of the inner surfaces 139 of the compression bearing 113 may be shaped and/or textured to limit a range of motion of the collar 112 relative to a bone fastener of a bone fastener assembly.

As used herein, the term “collar” includes any element that wholly or partially encloses or receives one or more other elements. A collar may enclose or receive elements including, but not limited to, a bone fastener, a closure member, a ring, a bearing and/or an elongated member. In some embodiments, a collar may couple two or more other elements together (e.g., an elongated member and a bone fastener). A collar may have any of various physical forms. In some embodiments, a collar may have a “U” shape, however it is to be understood that a collar may also have other shapes.

A collar may be open or closed. A collar having a slot and an open top, such as collar 112 shown in FIG. 2 and in FIG. 5, may be referred to as an “open collar.” A bone fastener assembly that includes an open collar may be referred to as an “open fastener.” In some embodiments, an elongated member may be top loaded into the open fastener. A closure member may be coupled to the collar to secure the elongated member to the open fastener.

A bone fastener may be rotatably positioned in a collar such that the bone fastener is able to move radially and/or rotationally relative to the collar (or the collar relative to the bone fastener) within a defined range of motion. The range of motion may be provided within a plane, such as by a hinged connection, or within a three-dimensional region, such as by a ball and socket connection. Motion of the bone fastener relative to the collar (or the collar relative to the bone fastener) may be referred to as “angulation” and/or “polyaxial movement”.

Collar 112 may include body 140 and arms 142. Arms 142 may extend from body 140. Body 140 of collar 112 may be greater in width than a width across arms 142 of collar 112 (i.e., body 140 may have a maximum effective outer diameter greater than a maximum effective outer diameter of arms 142). A reduced width across arms 142 may allow a detachable member to be coupled to the arms without substantially increasing a maximum effective outer diameter along a length of collar 112. Thus, a reduced width across arms 142 may reduce bulk at a surgical site.

A height of body 140 may range from about 3 millimeters (mm) to about 7 mm. In an embodiment, a height of body 140 is about 5 mm. Body 140 may include opening 144 in a lower surface of the body. To inhibit passage of a ring from collar 112, opening 144 may be smaller than an outer diameter of the ring. Inner surface 146 may be machined to complement a portion of an outer surface of a ring that is to be positioned in collar 112. Machining of inner surface 146 may enhance retention of a ring in collar 112. Inner surface 146 of body 140 may be complementary in shape to a portion of outer surface 132 of swivel bearing 110 (see FIG. 4A), stationary bearing 112 (see FIG. 4B) or compression bearing 113 (see FIG. 4C) so that the bearing is able to swivel in the collar. Inner surfaces and/or outer surfaces of collar 112 may be surface treated or include coatings and/or coverings to modify frictional properties or other properties of the collar. For example, inner surface 146 may be polished or coated with material such as Ultra High Molecular Weight Polyethylene (UHMWPE) or polyetheretherketone(PEEK) to provide dampened polyaxial motion of collar 112 relative to bone fastener 108.

Inner surfaces of arms 142 may include modified thread 148. Modified threads 148 may engage complementary modified threads of a closure member to secure an elongated member to a bone fastener assembly. Modified threads 148 may have a constant pitch or a variable pitch.

A height and a width of arms 142 may vary. Arms 142 may range in height from about 8 mm to about 15 mm. In an embodiment, a height of arms 142 is about 11 mm. A width (i.e., effective diameter) of arms 142 may range from about 5 mm to 14 cm. Arms 142 and body 140 may form slot 150. Slot 150 may be sized to receive an elongated member. Slot 150 may include, but is not limited to, an elongated opening of constant width, an elongated opening of variable width, a rectangular opening, a trapezoidal opening, a circular opening, a square opening, an ovoid opening, an egg-shaped opening, a tapered opening, and combinations and/or portions thereof In some embodiments, a first portion of slot 150 may have different dimensions than a second portion of slot 150. In certain embodiments, a portion of slot 150 in first arm 142 may have different dimensions than a portion of slot 150 in second arm 142. When an elongated member is positioned in slot 150, a portion of the elongated member may contact a head of a bone fastener positioned in the collar.

In some embodiments slot 150 may be configured with a depth essentially equal to the top of bone fastener 108 such that elongated member 104 may contact both bone fastener 108 and slot 150. In other embodiments, slot 150 may be configured, such as by machining, to a depth lower than the top of bone fastener 108 such that elongated member 104 may be supported or in contact with bone fastener 108 only. Machining refers generally to material processes for removing material and may include boring, drilling, milling and other machining processes for removing material from collar 112 such that a selected depth and profile are achieved.

In an embodiment of a collar, arms 142 of collar 112 may include one or more openings and/or indentions 152. Indentions 152 may vary in size and shape (e.g., circular, triangular, rectangular). Indentions 152 may be position markers and/or force application regions for instruments that perform reduction, compression, or distraction of adjacent vertebrae. In some embodiments, openings and/or indentions may be positioned in the body of the collar.

Arms 142 may include ridges or flanges 154. Flange 154 may allow collar 112 to be coupled to a detachable member so that translational motion of the collar relative to the detachable member is inhibited. Flanges 154 may also include notches 156. A movable member of a detachable member may extend into notch 156. When the movable member is positioned in notch 156, a channel in the detachable member may align with a slot in collar 112. With the movable member positioned in notch 156, rotational movement of collar 112 relative to the detachable member may be inhibited.

FIG. 6 depicts a cross-sectional representation of one embodiment of a bone fastener assembly with a friction reducing member. Bone fastener assembly 102 may include bone fastener 108, swivel ring 110, and collar 112. Bone fastener 108 of bone fastener assembly 102 may include passage 114. Bone fastener 108 may be cannulated (i.e., passage 114 may run through the full length of the bone fastener). A guide wire may be placed through passage 114 so that bone fastener 108 may be inserted into a vertebra at a desired location and in a desired angular orientation relative to the vertebra with limited or no visibility of the vertebra

In this configuration, collar 112 may exhibit polyaxial motion relative to swivel bearing 110. Swivel bearing 110 may rotate generally about the longitudinal axis of bone fastener 108. Alternatively, swivel bearing 110 may be in rigid contact with bone fastener such that all polyaxial motion is possible due to the motion allowed by the contact between swivel bearing 110 and collar 112.

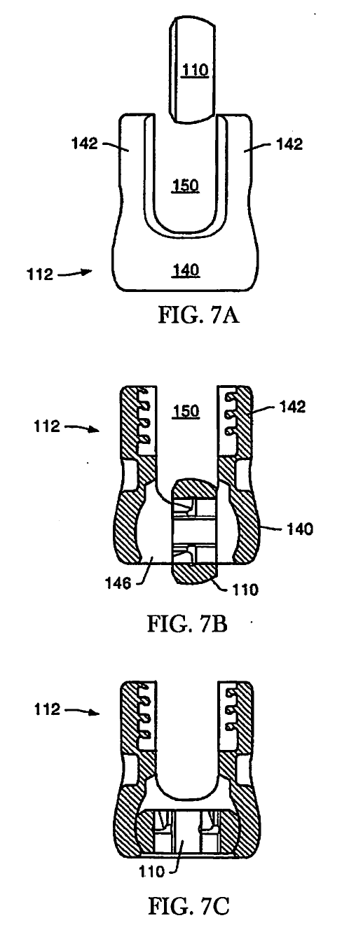

FIGS. 7A-7C show views of collar 112 and a friction reducing member (such as swivel bearing 110, stationary bearing 111, or bottom portion of compression bearing 113) during top loading insertion of the friction reducing member into the collar. Swivel bearing 110, stationary bearing, and at least a bottom portion of compression bearing 113 may be positioned as shown in FIG. 7A and inserted past arms 142 into body 140. In some embodiments (not shown) all of compression bearing 113 may be top loaded before a bone fastener may be loaded. FIG. 7B depicts a cross-sectional view of swivel bearing 110 and collar 112 after insertion of the swivel bearing into the collar through slot 150. After insertion of swivel bearing 110 into collar 112, swivel bearing 110 may be rotated so that a bone fastener may be positioned through the swivel bearing. FIG. 7C depicts a cross-sectional view of swivel bearing 110 and collar 112 after rotation of the bearing in the collar. Stationary bearing 111 and compression bearing 113 may be inserted into collar 112 similarly.

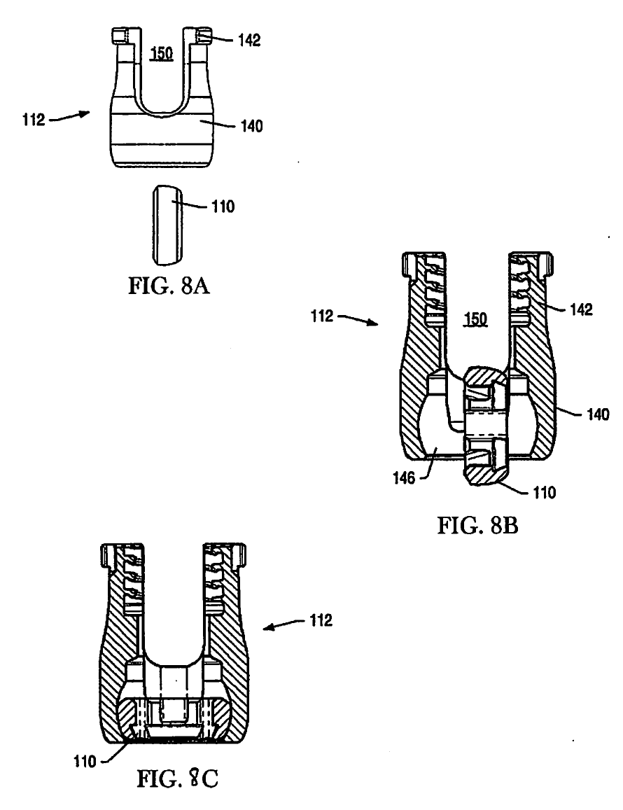

FIGS. 8A-8C show views of collar 112 and a friction reducing member such as swivel bearing 110, a stationary bearing (such as stationary bearing 111 in FIG. 4B), or a compression bearing (such as compression bearing 113 in FIG. 4C) during bottom loading insertion of the bearing into the collar. Swivel bearing 110 may be positioned as shown in FIG. 8A and inserted into body 140 through an opening in the bottom of collar 112. Stationary bearing 111 and compression bearing 113 may be inserted into collar 112 similarly. In some embodiments, a friction reducing member may be inserted into body 140 through a groove or a slot in the bottom of collar 112. In certain embodiments, collar 112 designed for bottom insertion of a friction reducing member may have narrower slot 150 than a collar designed for top insertion of the bearing. Collar 112 with narrower slot 150 may allow an elongated member with a reduced diameter to be used in a spinal stabilization system. Collar 112 with narrower slot 150 may be used to reduce bulk at a surgical site. FIG. 8B depicts a cross-sectional view of a friction reducing member such as a swivel bearing 110 and collar 112 after insertion of the bearing into the collar through the opening in the bottom of the collar. After insertion of the bearing into collar 112, the bearing may be rotated so that a bone fastener may be positioned through the bearing. Tolerance between an outer surface of bearing and an inner surface of body 140 shown in FIGS. 7A-7C and 8A-8C may require force to be applied to the bearing to drive the bearing into the body. Once the bearing is positioned in body 140, the bearing may expand slightly. In certain embodiments, significant force may be required to remove swivel bearing 110 from body 140 (i.e., the bearing may be substantially unreleasable from the body). The required force may inhibit unintentional removal of the bearing from body 140. FIG. 8C depicts a cross-sectional view of swivel bearing 110 and collar 112 after rotation of the swivel bearing 110 in the collar.

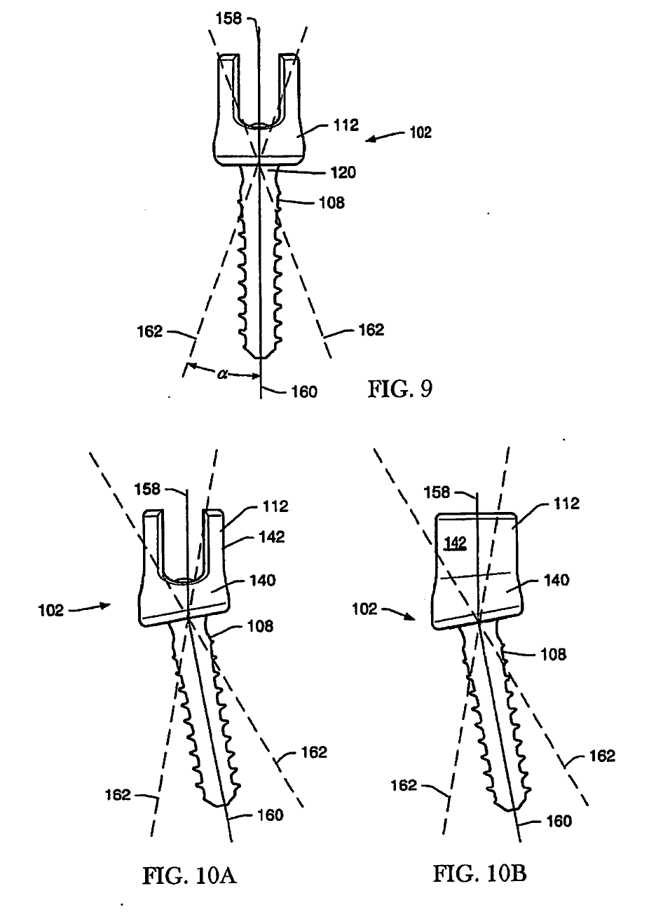

FIG. 9 depicts bone fastener assembly 102 with central axis 158 of collar 112 aligned with central axis 160 of bone fastener 108. Bone fastener 108 may be angulated in a symmetrical conical range of motion characterized by angle a about the aligned axes. Bone fastener 108 may be constrained from motion outside of limit axis 162 by contact between neck 120 of bone fastener 108 and collar 112. Alignment of axis 160 of bone fastener 108 with central axis 158 of collar 112 may be considered a neutral position relative to the range of motion. The alignment is a neutral position because bone fastener 108 may be angulated an equal amount in any direction from central axis 158. When a driver is inserted into bone fastener 108, axis 160 of bone fastener 108 may be substantially aligned with axis 158 of collar 112 to facilitate insertion of the bone fastener into a vertebral body.

In certain embodiments, a range of motion of a collar may be skewed from a full conical range of motion relative to aligned central axes of the collar and a bone fastener coupled to the collar. In some embodiments, a distal end of a collar may be shaped to skew, or bias, the range of motion from the range of motion depicted in FIG. 9.

FIGS. 10A and 10B depict bone fastener assemblies 102 with biased collars 112. Body 140 of biased collar 112 may be shaped to restrict relative movement of bone fastener 108 (and/or the collar) to a skewed conical range of motion defined by limit axes 162. As depicted by limit axes 162 in FIG. 10A, a first arm 142 of collar 112 may approach bone fastener 108 more closely than a second arm of the collar. As suggested by limit axes 162 in FIG. 10B, a first opening of the slot between arms 142 of collar 112 may approach bone fastener 108 more closely than a second opening of the slot.

Other biased collars may be designed to selectively restrict relative movement of collars and/or bone fasteners. In some embodiments, a biased collar may be attached to a detachable member such that a surgeon performing a minimally invasive procedure may selectively align the portion of the collar with the greater range of motion as needed. For example, the collar depicted in FIG. 10B may be coupled to a single-level (e.g., C-shaped) sleeve so that the side of the collar (i.e., the side of the slot) with a larger range of motion is positioned next to a channel opening of the sleeve.

When a biased collar of a bone fastener assembly is coupled to a detachable member and a drive mechanism is coupled to a bone fastener of the bone fastener assembly, central axis 158 of collar 112 may align with central axis 160 of bone fastener 108 to facilitate insertion of the bone fastener into bone. In some embodiments, the bias of the collar may be so large that a flexible drive member is needed to drive the bone fastener into bone.

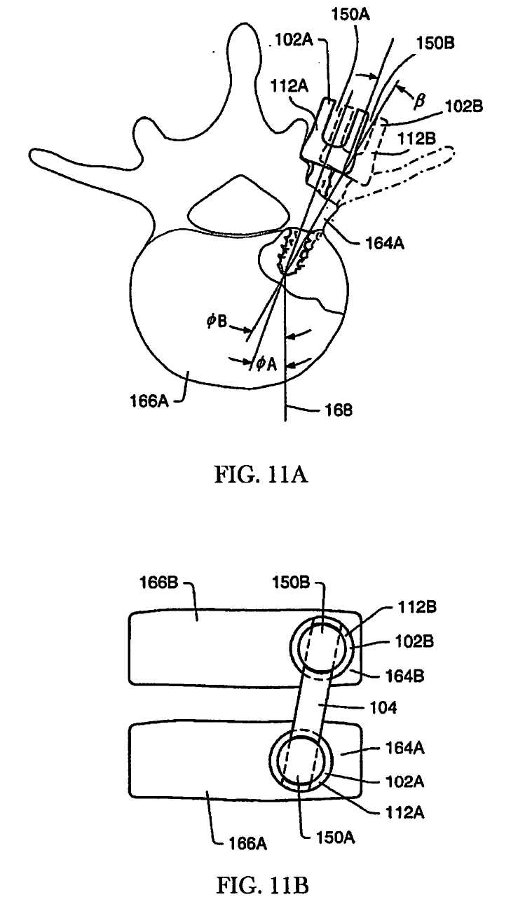

In some embodiments, one or more biased collars may be used in a spinal stabilization system. The spinal stabilization systems may be single-level systems or multi-level systems. Biased collars may be used to accommodate the increasing angle of the pedicle corridor for each lumbar vertebra. The angle may increase by about 5 degrees for each successive lumbar vertebra. FIGS. 11A and 11B depict a single-level spinal stabilization system including bone fastener assembly 102A coupled to pedicle 164A and vertebra 166A and bone fastener assembly 102B coupled to pedicle 164B and vertebra 166B.

A bone fastener of bone fastener assembly 102A may engage pedicle 164A at pedicle angle (phi-A) relative to sagittal plane 168. Pedicle angle (phi-A) may range between about 13 degrees and about 17 degrees. Collar 112A of bone fastener assembly 102A may be unbiased. Pedicle angle (phi-B) may range between about 18 degrees and about 22 degrees. Collar 112B may have a bias angle (beta) of about 5 degrees. Bone fastener assembly 102B may engage pedicle 164B at pedicle angle (phi-B) Because the bias of collar 112B is approximately equal to the difference between the pedicle angles of the two vertebrae, slots 150A and 150B in bone fastener assemblies 102A and 102B, respectively, may be generally aligned when both bone fasteners are in neutral positions.

Angulation of either or both collars of the bone fastener assemblies may allow fine adjustment of engagement angles of the bone fasteners. In addition, collar angulation may allow adjustment in the orientation of bone fasteners in a sagittal plane (i.e., to conform to lordosis of a spine) while still allowing the collars to be easily coupled with elongated member 104. Elongated member 104 may be disposed in slots 150A and 150B and secured by closure members. In some embodiments, a flexible driver or a polyaxial driver (e.g., a driver with a universal joint) may be used to drive the heads of the bone fasteners from a position that is off-axis from the bone fasteners to reduce the size of an opening of the body needed to implant the spinal stabilization system.

A closure member may be coupled to a collar of a bone fastener assembly to fix an elongated member positioned in the collar to the bone fastener assembly. In some embodiments, a closure member may be cannulated. In certain embodiments, a closure member may have a solid central core. A closure member with a solid central core may allow more contact area between the closure member and a driver used to couple the closure member to the collar. A closure member with a solid central core may provide a more secure connection to an elongated member than a cannulated closure member by providing contact against the elongated member at a central portion of the closure member as well as near an edge of the closure member. FIG. 1 depicts closure members 106 coupled to bone fastener assemblies 102.

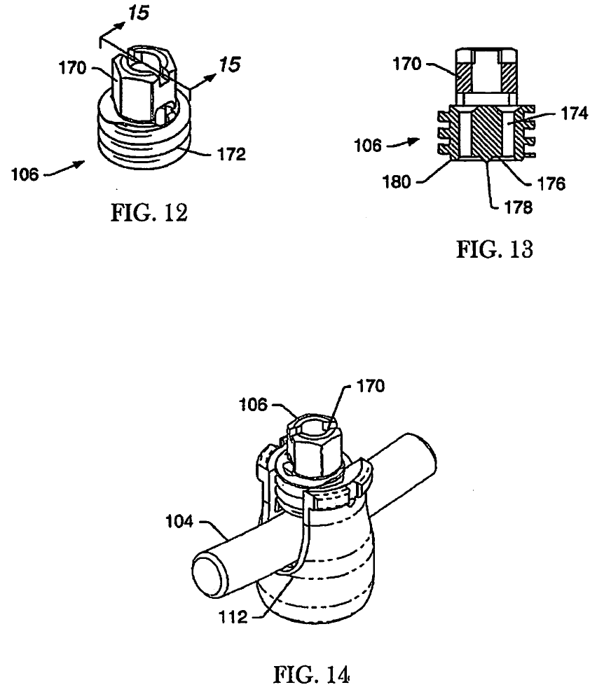

FIGS. 12 and 13 depict closure member 106 prior to insertion of the closure member into a collar of a bone fastener assembly. Closure member 106 may include tool portion 170 and male modified thread 172. Tool portion 170 may couple to a tool that allows closure member 106 to be positioned in a collar. Tool portion 170 may include various configurations (e.g., threads, hexalobular connections, hexes) for engaging a tool (e.g., a driver). Male modified thread 172 may have a shape that complements the shape of a female modified thread in arms of a collar (e.g., modified thread 148 depicted in FIG. 5). A secure connection to collar 112 may be achieved by mechanical, chemical, or thermal processes or devices. For example, in the embodiment shown in FIG. 5, slot 150 in collar 112 has a discontinuous helically wound thread for threadably engaging a corresponding external thread on closure member 106. In other embodiments, not shown, slot 150 may have a stepped diameter profile that limits how deep a threaded insert may penetrate collar 112. Advantageously, having a stepped diameter creates a shoulder in collar 112 that prevents a threaded insert, such as closure member 106 from impinging a elongated member and preventing motion between bone fastener 108 and elongated member 104.

Although closure member 106 is depicted in these figures as having a thread, the present disclosure is not so limited, and closure member 106 may also be glued, epoxied, or otherwise chemically connected to collar 112 or may be sweat-locked or otherwise thermally connected to collar 112 to provide a secure connection that allows polyaxial motion.

FIG. 14 depicts a portion of a spinal stabilization system with closure member 106 coupled to collar 112 before tool portion 170 is sheared off. Closure member 106 may couple to collar 112 by a variety of systems including, but not limited to, standard threads, modified threads, reverse angle threads, buttress threads, or helical flanges. A buttress thread on a closure member may include a rearward-facing surface that is substantially perpendicular to the axis of the closure member. Closure member 106 may be advanced into an opening in a collar to engage a portion of elongated member 104. In some embodiments, closure member 106 may inhibit movement of elongated member 104 relative to collar 112.

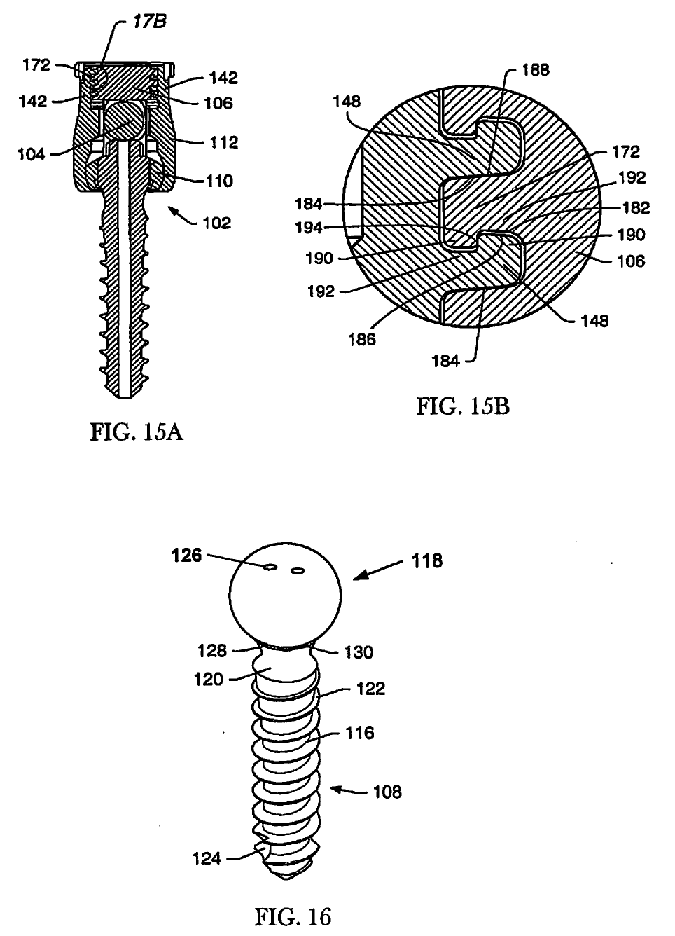

FIG. 15A depicts a cross-sectional view of closure member 106 coupled to bone fastener assembly 102. Closure member 106 may include male modified thread 172. Male modified thread 172 may include male distal surface 182 and male proximal surface 184, as shown in FIG. 15B. Collar 112 may include female modified thread 148 on an inside surface of arms 142. Female modified thread 148 may include female proximal surface 186 and female distal surface 188. Male proximal surface 184 may couple to female distal surface 188 during use. Male proximal surface 184 and female distal surface 188 may be load-bearing surfaces. A load may result from an upward load on closure member 106, such as a load resulting when elongated member 104 positioned in a slot of collar 112 is secured to bone fastener assembly 102 by closure member 106.

In an embodiment, a bone fastener assembly and a closure member may be coupled with a running fit. A running fit (i.e., a fit in which parts are free to rotate) may result in predictable loading characteristics of a coupling of a bone fastener assembly and a closure member. Predictable loading characteristics may facilitate use of a closure member with a break-off portion designed to shear off at a predetermined torque. A running fit may also facilitate removal and replacement of closure members. In some embodiments, a closure member may include an interference fit (e.g., crest-to-root radial interference).

In an embodiment, a position (i.e., axial position and angular orientation) of a modified thread of a collar may be controlled, or “timed,” relative to selected surfaces of the collar. For example, a modified thread form may be controlled relative to a top surface of a collar and an angular orientation of the slots of the collar. In some embodiments, positions of engaging structural elements of other coupling systems (e.g., thread forms) may be controlled.

Controlling a position of a modified thread form may affect a thickness of a top modified thread portion of a collar. In FIG. 5, top modified thread portion 196 is the first modified thread portion to engage a closure member. In an embodiment, a position of a modified thread form may be selected such that the thickness of the leading edge of a top modified thread portion is substantially equal to the full thickness of the rest of the modified thread.

Controlling a position of a modified thread form of a collar may increase a combined strength of engaged modified thread portions for a collar of a given size (e.g., wall height, modified thread dimensions, and thread pitch). Controlling a position of the modified thread form may reduce a probability of failure of modified thread portions, and thus reduce a probability of coupling failure between a collar and a closure member. Controlling the position of a modified thread form in a collar of a bone fastener assembly may increase a combined strength of engaged collar and closure member modified thread portions such that failure of the modified thread portions does not occur prior to the intended shearing off of a tool portion of the closure member. For example, a tool portion of a closure member may be designed to shear off at about 90 in-lbs of torque, while the combined modified thread portions may be designed to withstand a torque on the closure member of at least 120 in-lbs.

If a thickness of a modified thread portion of a given size and profile is reduced below a minimum thickness, the modified thread portion may not significantly contribute to the holding strength of the modified thread of a collar. In an embodiment, a position of a modified thread form of a collar may be controlled such that a thickness of a top modified thread portion is sufficient for the portion to increase a holding strength of the collar. In one embodiment, a top modified thread portion may have a leading edge thickness of about 0.2 mm.

In an embodiment, a position of a modified thread form of a collar may be selected to ensure that a closure member engages a selected minimum number of modified thread portions on each arm of the collar. In an embodiment, at least two modified thread portions having a full thickness over width w of a collar arm (shown in FIG. 5) may be engaged by a closure member at each arm. Alternatively, a closure member may engage parts of three or more modified thread portions on each arm, with the total width of the portions equal to at least two full-width portions. Allowances may be made for tolerances in the components (e.g., diameter of the elongated member) and/or anticipated misalignment between the components, such as misalignment between an elongated member and a slot. In an embodiment, a substantially equal number of modified thread portions in each arm may engage the closure member when an elongated member is coupled to a bone fastener assembly.

Minimally invasive procedures may involve locating a surgical site and a position for a single skin incision to access the surgical site. The incision may be located above and between (e.g., centrally between) vertebrae to be stabilized. An opening under the skin may be enlarged to exceed the size of the skin incision. Movement and/or stretching of the incision, bending of an elongated member, and angulation of collars of bone fastener assemblies may allow the length of the incision and/or the area of a tissue plane to be minimized. In some embodiments, minimally invasive insertion of a spinal stabilization system may not be visualized. In certain embodiments, insertion of a spinal stabilization system may be a top-loading, mini-opening, muscle-splitting, screw fixation technique.

A bone fastener assembly with a bone fastener of an appropriate length may be selected for insertion in a patient. The size of the bone fastener may be verified with measurement indicia in an instrumentation set. In some embodiments, measurement indicia may be etched or printed on a portion of an instrumentation set. For example, the chosen bone fastener embodiment may be placed over the outline of a bone fastener embodiment printed on a tray of the instrumentation set.

The chosen bone fastener assembly may be attached to a detachable member. When the bone fastener assembly is coupled to the detachable member, a drive portion of a fastener driver may be coupled to a tool portion of the bone fastener. A shaft of the fastener driver may be positioned in the passage of the detachable member. A removable handle may be attached to the shaft of the fastener driver. The detachable member, collar, and bone fastener may be substantially co-axial when the fastener driver is positioned in the detachable member. In some embodiments, the removable handle may be attached to the shaft of the fastener driver after the bone fastener, collar, detachable member, and fastener driver combination is positioned down a guide wire through a dilator and against a pedicle.

After insertion of the bone fastener assembly, the driver may be rotated to thread the bone fastener into a pedicle in a vertebral body. The bone fastener may be advanced into the pedicle under fluoroscopic guidance to inhibit breaching of the pedicle walls. When the tip of the bone fastener advances beyond the posterior margin of the vertebral body, a guide wire may be removed to inhibit inadvertent bending of the guide wire or unwanted advancement of the guide wire.

The bone fastener may be advanced to bring the collar down snug to the facet joint. The bone fastener may then be backed off about a quarter of a turn. Backing the fastener off about a quarter of a turn may allow for full motion of the collar relative to the bone fastener. After the bone fastener has been advanced to the desired depth, the driver may be removed from the head of the bone fastener.

After the bone fastener has been secured to the vertebra and the driver has been removed from the sleeve, the polyaxial nature of the friction reducing member may allow angulation of the collar relative to the bone fastener. With bone fastener assemblies secured in the vertebral bodies, sleeves coupled to the bone fastener assemblies may be oriented to facilitate insertion of an elongated member in the sleeves. In some embodiments, sleeves may serve as tissue retractors during a spinal stabilization procedure. Angular motion of a collar may be limited by a range of motion allowed between the collar and the bone fastener that the collar is anchored to. Angular motion of a collar may be limited by patient anatomy. Angular motion or orientation of one collar, however, may not depend upon a position of another collar.

An elongated member may be cut to length and contoured as desired. For example, a medical practitioner may use experience and judgment to determine curvature of an elongated member for a patient. A desired curvature for the elongated member may be determined using fluoroscopic imaging. The elongated member may be bent or shaped with a tool (e.g., a rod bender) to allow insertion of the elongated member through channels of sleeves with various spatial locations and/or various angular orientations. Elongated members may have shapes including, but not limited to, straight, bent, curved, s-shaped, and z-shaped.

In some embodiments, elongated members may have a substantially circular longitudinal cross section. In certain embodiments, elongated members may have other cross-sectional shapes including, but not limited to, regular shapes (oval, rectangular, rhomboidal, square) and irregular shapes. An instrumentation kit for a spinal stabilization system may include straight rods and/or pre-shaped rods. Straight rods and/or pre-shaped rods may be contoured to accommodate patient anatomy if needed during the surgical procedure.

After the elongated member is seated in the collars, additional fluoroscopic confirmation of elongated member positioning may be obtained. With the elongated member satisfactorily positioned, the elongated member may be secured in place with closure members.

The closure member may secure the elongated member to the collar. When the closure members are snug and the elongated member is secured, collars may be angled such that slots in the collars are substantially perpendicular to the elongated member.

After a closure member is successfully secured to a collar, the driver may be removed from the sleeve coupled to the anchored bone fastener assembly.

A spinal stabilization system may be used to stabilize two or more vertebral levels (i.e., at least three adjacent vertebrae). In an embodiment, an incision may be made in the skin between the outermost vertebrae to be stabilized. A first bone fastener assembly may be coupled to a first sleeve. The first bone fastener may be threaded into a first pedicle at a target location such that the first sleeve extends above the body surface. The first sleeve may rotate about the head of the first bone fastener. A tissue plane may be created between a channel opening in the first sleeve and a target location at a second pedicle. In an embodiment, the second pedicle may be adjacent to the first pedicle. A second bone fastener assembly may be coupled to a second sleeve and threaded into the second pedicle through the incision. Another tissue plane may be created between the first sleeve or the second sleeve and a target location in a third pedicle. The third pedicle may be adjacent to the first pedicle and/or the second pedicle. A third bone fastener assembly may be coupled to a third sleeve and threaded into the third pedicle through the incision.

In an embodiment of a method for a two-level spinal stabilization procedure, an incision may be made above a target location in a middle pedicle. A first bone fastener may be anchored to the middle pedicle. After the first bone fastener is secured, second and third bone fasteners may be coupled to outer pedicles as desired by pulling and/or stretching tissue surrounding the incision to allow access to the outer pedicles.

In some embodiments, a spinal stabilization system may be inserted using an invasive procedure. Since insertion of a spinal stabilization system in an invasive procedure may be visualized, cannulated components (e.g., bone fasteners) and/or instruments (e.g., detachable members) may not be needed for the invasive (i.e., open) procedure. Thus, a bone fastener used in an invasive procedure may differ from a bone fastener used in a minimally invasive procedure. FIG. 16 depicts a perspective view of an embodiment of bone fastener 108 that may be used in an invasive procedure.

Bone fastener 108 may include shank 116, head 118, and neck 120. Shank 116 may include threading 122. In some embodiments, threading 122 may include self-tapping start 124. Self-tapping start 124 may facilitate insertion of bone fastener 108 into vertebral bone. Head 118 of bone fastener 108 may be generally spherical and may also include various configurations to engage a driver that inserts the bone fastener into a vertebra. In certain embodiments, the driver may also be used to remove an installed bone fastener from a vertebra.

In some embodiments, head 118 may include one or more tool portions 126. Tool portions 126 may be recesses and/or protrusions designed to engage a portion of the driver. In certain embodiments, bone fasteners with closed collars may be used in an invasive spinal stabilization procedure. In certain embodiments, fixed bone fasteners (e.g., open fixed bone fasteners) may be used in an invasive spinal stabilization procedure.

In some embodiments, tools used in an invasive procedure may be similar to tools used in a minimally invasive procedure. In certain embodiments, methods of installing a spinal stabilization system in an invasive procedure may be similar to methods of installing a spinal stabilization system in a minimally invasive procedure.

Further modifications and alternative embodiments of various aspects of the disclosure will be apparent to those skilled in the art in view of this description. Accordingly, this description is to be construed as illustrative only and is for the purpose of teaching those skilled in the art the general manner of carrying out the disclosure. It is to be understood that the forms of the disclosure shown and described herein are to be taken as the presently preferred embodiments. Elements and materials may be substituted for those illustrated and described herein, parts and processes may be reversed, and certain features of the disclosure may be utilized independently, all as would be apparent to one skilled in the art after having the benefit of this description of the disclosure. Changes may be made in the elements described herein without departing from the spirit and scope of the disclosure as described in the following claims.

What is claimed is:

1. A friction reducing member for use in spine stabilization systems, comprising:
an inner surface configured to contour to a head of a bone fastener; and
an outer surface configured to contour to an opening of a collar in a bone fastener assembly,
wherein one or more of the inner surface and outer surface is configured for low friction coefficient.

2. The friction reducing member of claim 1, wherein the friction reducing member comprises ultra-high molecular weight polyethylene (UHMWPE).

3. The friction reducing member of claim 2, wherein the friction reducing member is a swivel bearing comprising
an inner surface configured for rotatable contact with the bone fastener; and
an outer surface configured for polyaxial contact between the collar and the swivel bearing.

4. The friction reducing member of claim 2, wherein the friction reducing member is a stationary bearing comprising
an outer surface configured for polyaxial contact relative to the inner surface of the collar; and
an inner surface configured for polyaxial contact relative to the bone fastener.

5. The friction reducing member of claim 2, wherein the friction reducing member is a compression bearing comprising:
an upper portion having an inner surface configured for polyaxial contact with a portion of a head of a bone fastener;
a lower portion having an inner surface configured for polyaxial contact with a portion of a head of a bone fastener,
wherein the outer surface of the upper and lower portions are configured for rotatable contact between the collar and the compression bearing.

6. A bone fastener assembly comprising:
a collar comprising:
an opening for receiving a portion of a bone fastener; and
a slot at least partially open to the opening and configured to receive at least a portion of an elongated member;
a closure member configured for selected contact with the elongated member and further configured for secure connection to the collar to retain the elongated member in the slot; and
a friction reducing member disposed between at least a portion of the opening and at least a portion of the bone fastener and configured for selected contact such that dampened polyaxial motion of the elongated member relative to the bone fastener is preserved when the closure member is securely connected to the collar.

7. The bone fastener assembly of claim 6, wherein the collar further comprises a channel having a cylindrical inner surface with modified thread portion, and the closure member is configured with a helically wound thread for rotatably engaging the modified thread portion.

8. The bone fastener assembly of claim 8, wherein the closure member comprises a layer having a low friction coefficient for selected contact with the elongated member.

9. The bone fastener assembly of claim 7, wherein the layer comprises ultra-high molecular weight polyethylene (UHMWPE).

10. A dynamic stabilization system, comprising:
two or more bone fasteners implantable in bony tissue;
an elongated member of selected length to span between the two or more bone fasteners implanted in bony tissue; and
two or more collars for connecting the elongated member to the two or more bone fasteners, each collar comprising:
an opening for receiving a portion of a bone fastener; and
a slot at least partially open to the opening and configured to receive at least a portion of a elongated member;
a closure member configured for selected contact with the elongated member and further configured for secure connection to the collar to retain the elongated member in the slot; and
a friction reducing member disposed between a portion of the opening and a portion of the bone fastener and configured for selected contact such that dampened polyaxial motion of the elongated member relative to the bone fastener is preserved when the closure member is securely connected to the collar.

11. The collar of claim 10, wherein the friction reducing member comprises ultra-high molecular weight polyethylene (UHMWPE).

12. The collar of claim 10, wherein the friction reducing member comprises a swivel bearing comprising:
an inner surface configured for rotatable contact with the bone fastener; and
an outer surface configured for polyaxial contact between the collar and the swivel bearing.

13. The collar of claim 10, wherein the friction reducing member comprises a stationary bearing comprising:
an outer surface configured for polyaxial contact relative to the inner surface of the collar; and
an inner surface configured for polyaxial contact relative to the bone fastener.

13. The collar of claim 9, wherein the friction reducing member comprises a compression bearing comprising
an upper portion having an inner surface configured for polyaxial contact with a portion of a head of a bone fastener;
a lower portion having an inner surface configured for polyaxial contact with a portion of a head of a bone fastener,
wherein the outer surface of the upper and lower portions are configured for rotatable contact between the collar and the compression bearing.

14. The collar of claim 9, wherein the collar further comprises a channel having a cylindrical inner surface with modified thread portion, wherein the closure member is configured with a helically wound thread for rotatably engaging the modified thread portion.

15. A method for dynamically stabilizing a spine, comprising: coupling a collar to an bone fastener in a bony tissue, wherein each collar comprises:
an opening for receiving a portion of one of the two or more bone fasteners;
a slot at least partially open to the opening and configured to receive at least a portion of the elongated member;
a closure member configured for selected contact with the elongated member and further configured for secure connection to the collar to maintain the elongated member in the slot;
a friction reducing member disposed between at least a portion of the collar and at least a portion of the bone fastener and operable to provide selected friction resistance;
positioning a portion of a elongated member in the collar; and
connecting the closure member to the collar to maintain the collar in movable contact with the bone fastener, wherein the collar is configured such that dampened polyaxial motion of the elongated member relative to the bone fastener is preserved when the closure member is securely connected to the collar.

16. The method of claim 15, wherein the step of connecting the closure member to the collar to maintain the collar in movable contact with the bone fastener comprises rotatably engaging the closure member having a helically wound thread with a modified thread portion.

17. The method of claim 15, further comprising the step of positioning a friction reducing member inside the collar, wherein the friction reducing member is selected for low friction coefficient.

18. The collar of claim 17, wherein the friction reducing member comprises ultra-high molecular weight polyethylene (UHMWPE).

19. The collar of claim 18, wherein the friction reducing member is a swivel bearing comprising
an inner surface configured for rotatable contact with the bone fastener; and
an outer surface configured for polyaxial contact between the collar and the swivel bearing.

20. The collar of claim 18, wherein the friction reducing member comprises a stationary bearing comprising
an outer surface configured for polyaxial contact relative to the inner surface of the collar; and
an inner surface configured for polyaxial contact relative to the bone fastener.

21. The collar of claim 18, wherein the friction reducing member comprises a compression bearing comprising
an upper portion having an inner surface configured for polyaxial contact with a portion of a head of a bone fastener;
a lower portion having an inner surface configured for polyaxial contact with a portion of a head of a bone fastener,
wherein the outer surface of the upper and lower portions are configured for rotatable contact between the collar and the compression bearing.
